(19) United States
(12) Patent Application Publication
(10) Pub. No.: US 2026/0345882 A1
(43) Pub. Date: Nov. 5, 2026

(54) SYSTEM FOR PRODUCING DIALYSATE AND QUANTIFYING GLUCOSE IN SPENT DIALYSATE

(71) Applicant: Byonyks Medical Devices, Inc., Itasca, IL (US)

(72) Inventor: Rodney S. KENLEY, Itasca, IL (US)

(73) Assignee: Byonyks Medical Devices, Inc., Itasca, IL (US)

(21) Appl. No.: 19/478,324

(22) PCT Filed: Apr. 26, 2024
(86) PCT No.: PCT/US2024/026405

§ 371 Date: Oct. 24, 2025

Related U.S. Application Data
(60) Provisional application No. 63/498,685, filed on Apr. 27, 2023.

(51) Int. Cl. A61M 1/16; A61M 1/26; A61M 1/36

(52) U.S. Cl. CPC A61M 1/1609; A61M 1/1668; A61M 1/1672; A61M 1/267; A61M 1/3661; A61M 2205/3306

(57) ABSTRACT

In a fluorometer diagnostic mode, a system moves water and a fluorescent probe material from a mixing chamber to a fluorometer. In a filter diagnostic mode, the system moves water and a fluorescent probe material from the mixing chamber into an inlet such that (i) a first portion of the water and the fluorescent probe material moves out of a bypass outlet and back to the mixing chamber and (ii) a second portion of the water moves through the filter to the fluorometer. In a dialysate dispensing mode, the system moves a dialysate and the fluorescent probe material from the mixing chamber into the inlet such that (i) a first portion of the dialysate and the fluorescent probe material moves out of the bypass outlet and back to the mixing chamber and (ii) a second portion of the dialysate moves through the filter and to the fluorometer.

### CROSS-REFERENCE TO RELATED APPLICATIONS

This application claims priority to U.S. Provisional Patent Application No. 63/498,685, filed on Apr. 27, 2023, the entire contents of which are incorporated by reference herein.

This application incorporates by reference the entire contents of international application No. PCT/US22/82269, filed on Dec. 22, 2022.

### BACKGROUND

There are two principal dialysis methods used to support patients requiring renal replacement therapy: hemodialysis and peritoneal dialysis. Peritoneal dialysis utilizes the patient's own peritoneum as a semipermeable membrane. The peritoneum is the membranous lining of the body cavity surrounding all of the organs between the diaphragm and the pelvis that, due to the large number of blood vessels and capillaries imbedded therein, is capable of acting as a natural semipermeable membrane.

In peritoneal dialysis, a sterile dialysate is infused into the peritoneal cavity by way of an indwelling catheter. This can be accomplished manually by gravity or with the use of a machine known as a cycler. An osmotic pressure gradient is generated by including an osmotic agent in the peritoneal dialysate. The osmotic agent used in the vast majority of peritoneal dialysate is glucose. The dialysate is allowed to dwell in the peritoneal cavity for a sufficient length of time (e.g. 4 hours) to yield a net removal of toxins and water after which the dialysate is drained and replaced with fresh dialysate.

There are two primary forms of peritoneal dialysis (PD): Continuous Ambulatory Peritoneal Dialysis (CAPD) and Continuous Cycling Peritoneal Dialysis (CCPD). With CCPD, fluid exchanges can be performed while the patient is sleeping, with the inflow and outflow of dialysate controlled by a cycler. This is designed to spare the patient from the drudgery of performing these exchanges during their waking hours. Typically, the cycler leaves them with a final fill volume just before they wake up and this is left in the peritoneum until midday when the patient performs a drain. The patient then has the option to remain “dry” until that night which has the benefits of letting their peritoneal membrane recover from the constant onslaught of low pH/high osmolarity solution, or, if they require more toxin and water removal, they can manually instill another fill volume for the rest of the day.

One disadvantage of CCPD is the burden of storing, connecting, disconnecting, and disposing supplies that are used during the process, such as sterilized bags of solution, tubing sets and connectors, and the ancillary supplies required to perform aseptic connections/disconnections.

Some devices are designed to produce injectable quality dialysate for providing renal replacement therapy on-line during a therapeutic session. Some of these methods employing cold filtration sterilization methods use at least two redundant depyrogenation filters (e.g., ultrafilters) in series to assure that only sterile and pyrogen-free dialysate will reach a patient even if there is a membrane leak in one of the filters during a dialysis treatment.

Dual ultrafilter designs require that both filters be tested for membrane integrity prior to each dialysis treatment (i.e., a T₀ test). Some designs have accomplished this by filling the filters with air, pressurizing them, and then tracking the rate of decay in the pressure with pressure sensors and determining whether the rate of decay falls within the safe window that correlates with intact membranes. Following this, the filters are then re-primed by replacing the air with fluid which can be difficult to accomplish without human intervention because micro-bubbles tend to stick to the surface of the membrane and need mechanical agitation to encourage their release. To the extent to which bubbles remain, the effective surface area of the membrane is reduced.

### SUMMARY

A first example includes a system for producing dialysate, the system comprising: a mixing chamber; a pump; a filter cartridge comprising a filter, an inlet, a bypass outlet, and a filtering outlet; a fluorometer; one or more valves; and one or more tubing lines, wherein the system is configured to operate in a fluorometer diagnostic mode, a filter diagnostic mode, and a dialysate dispensing mode, wherein in the fluorometer diagnostic mode the one or more valves and the one or more tubing lines are configured to allow the pump to move water and a fluorescent probe material from the mixing chamber, into the inlet, out of the bypass outlet, and to the fluorometer, wherein in the filter diagnostic mode the one or more valves and the one or more tubing lines are configured to allow the pump to move the water and the fluorescent probe material from the mixing chamber into the inlet such that (i) a first portion of the water and the fluorescent probe material moves out of the bypass outlet and back to the mixing chamber and (ii) a second portion of the water moves through the filter, out of the filtering outlet, and to the fluorometer, and wherein in the dialysate dispensing mode the one or more valves and the one or more tubing lines are configured to allow the pump to move a dialysate and the fluorescent probe material from the mixing chamber into the inlet such that (i) a first portion of the dialysate and the fluorescent probe material moves out of the bypass outlet and back to the mixing chamber and (ii) a second portion of the dialysate moves through the filter, out of the filtering outlet, and to the fluorometer.

A second example includes a method of operating the system of the first example, the method comprising moving a first portion of the water and a first portion of the fluorescent probe material from the mixing chamber, into the inlet, out of the bypass outlet, and to the fluorometer; detecting the first portion of the fluorescent probe material in the first portion of the water using the fluorometer; in response to the detecting, moving a second portion of the water and a second portion of the fluorescent probe material from the mixing chamber into the inlet such that the second portion of the water moves through the filter, out of the filtering outlet, and to the fluorometer; determining, using the fluorometer, that less than a first threshold amount of the second portion of the fluorescent probe material passed through the filter with the second portion of the water; in response to the determining, filling the mixing chamber with a dialysate and moving the dialysate and a third portion of the fluorescent probe material from the mixing chamber into the inlet such that the dialysate moves through the filter, out of the filtering outlet, and to the fluorometer; and dispensing the dialysate via a patient line in response to determining that less than a second threshold amount of the third portion of the fluorescent probe material passed through the filter with the dialysate; or dispensing the dialysate via a drain line in response to determining that more than the second threshold amount of the third portion of the fluorescent probe material passed through the filter with the dialysate.

A third example is a non-transitory computer readable medium storing instructions that, when executed by a computing device, cause the computing device to perform the method of the second example.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a block diagram of a system that includes a computing device, according to an example.

FIG. 2 is a schematic diagram of a system, according to an example.

FIG. 3 is a schematic diagram of a system configured for operation in a fluorometer diagnostic mode, according to an example.

FIG. 4 is a schematic diagram of a system configured for operation in a filter diagnostic mode, according to an example.

FIG. 5 is a schematic diagram of a system configured for operation in a dialysate production mode, according to an example.

FIG. 6 is a schematic diagram of a system configured for operation in a dialysate dispensing mode, according to an example.

FIG. 7 is a schematic diagram of a system configured for operation in a glucose quantification mode according to an example.

FIG. 8 is a block diagram of a method, according to an example.

FIG. 9 is a block diagram of a method, according to an example.

FIG. 10 is a schematic diagram of a system, according to an example.

FIG. 11 is a schematic diagram of a system, according to an example.

FIG. 12 is a data plot showing the relationship between the fluorescence of spent dialysate and its glucose concentration, according to an example.

### DETAILED DESCRIPTION

A significant improvement over conventional methods of establishing the patency of a depyrogenation filter would be one that could continuously monitor the absence of a fiber leak throughout a dialysis treatment and, by so doing, eliminate the need for a second filter as well as the need to perform an air pressure decay test. This disclosure is directed to a method of supporting the membrane integrity of a depyrogenation filter used in the preparation of injectable quality dialysate not just prior to the start of a dialysis treatment, but continuously throughout the treatment. This method can reduce the need for a redundant filter as well as reduce the need for performing an air pressure decay test or other test as a verification of membrane integrity followed by re-priming.

The second aspect of this disclosure is directed to a method of quantifying the amount of glucose in the dialysate being drained from a patient performing peritoneal dialysis. Glucose (Dextrose) is included in peritoneal dialysate as the osmotic agent that is responsible for drawing water out of a patient's blood to compensate for their inability to eliminate excess water via urination. During the dwell phase of a peritoneal dialysis treatment, at the same time water is moving from blood to dialysate, glucose is moving from dialysate to blood such that the concentration of glucose exiting the patient after a dwell period will be less than it was during instillation. This is a distinct drawback to the peritoneal dialysis modality. The absorbed glucose tends to reduce patients' appetite for protein, and they can become protein malnourished while at the same time gaining weight from the continuous absorption of calories from sugar. Many become obese as a result. Also, the absorbed glucose complicates the management of diabetes which is a significant concern given that over 50% of the incident dialysis population in the U.S. are diabetic.

Knowing the amount of sugar absorbed by a patient during a given treatment would be highly desirable for clinicians to help them prescribe the best Dextrose concentration to use in the dialysate as well as the amount of insulin to inject and when. This data could also be provided directly to continuous glucose monitoring devices used by many patients. As such, any peritoneal dialysis device that could provide a quantification of the absorbed glucose could confer a differential competitive advantage.

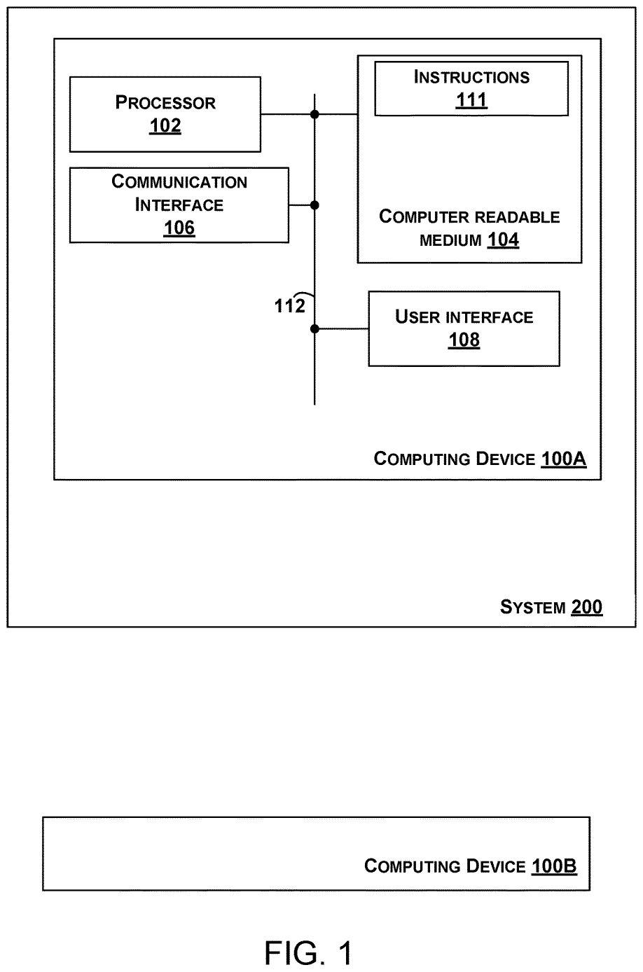

FIG. 1 is a block diagram of a system 200 for peritoneal dialysis. The system 200 includes a computing device 100A. In some examples, the computing device 100A directly controls the system 200, but in other examples, the computing device 100B can control the system 200 by sending instructions to the computing device 100A via a wired or wireless connection. For example, the computing device 100B can take the form of a tablet computer, a laptop computer, a smartphone, etc. The features and components of the computing device 100 described below can refer to the computing device 100A and/or the computing device 100B in various examples. Further features of the system 200 are detailed in subsequent Figures.

The computing device 100 includes one or more processors 102, a non-transitory computer readable medium 104, a communication interface 106, and a user interface 108. Components of the computing device 100 are linked together by a system bus, network, or other connection mechanism 112.

The one or more processors 102 can be any type of processor(s), such as a microprocessor, a field programmable gate array, a digital signal processor, a multicore processor, etc., coupled to the non-transitory computer readable medium 104.

The non-transitory computer readable medium 104 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 104 can store instructions 111. The instructions 111 are executable by the one or more processors 102 to cause the computing device 100 to perform any of the functions or methods described herein.

The communication interface 106 can include hardware to enable communication within the computing device 100 and/or between the computing device 100 and one or more other devices. The hardware can include any type of input and/or output interfaces, a universal serial bus (USB), PCI Express, transmitters, receivers, and antennas, for example. The communication interface 106 can be configured to facilitate communication with one or more other devices, in accordance with one or more wired or wireless communication protocols. For example, the communication interface 106 can be configured to facilitate wireless data communication for the computing device 100 according to one or more wireless communication standards, such as one or more Institute of Electrical and Electronics Engineers (IEEE) 801.11 standards, ZigBee standards, Bluetooth standards, etc. As another example, the communication interface 106 can be configured to facilitate wired data communication with one or more other devices. The communication interface 106 can also include analog-to-digital converters (ADCs) or digital-to-analog converters (DACs) that the computing device 100 can use to control various components of the computing device 100 or external devices.

The user interface 108 can include any type of display component configured to display data. As one example, the user interface 108 can include a touchscreen display. As another example, the user interface 108 can include a flat-panel display, such as a liquid-crystal display (LCD) or a light-emitting diode (LED) display. The user interface 108 can include one or more pieces of hardware used to provide data and control signals to the computing device 100. For instance, the user interface 108 can include a mouse or a pointing device, a keyboard or a keypad, a microphone, a touchpad, or a touchscreen, among other possible types of user input devices. Generally, the user interface 108 can enable an operator to interact with a graphical user interface (GUI) provided by the computing device 100 (e.g., displayed by the user interface 108).

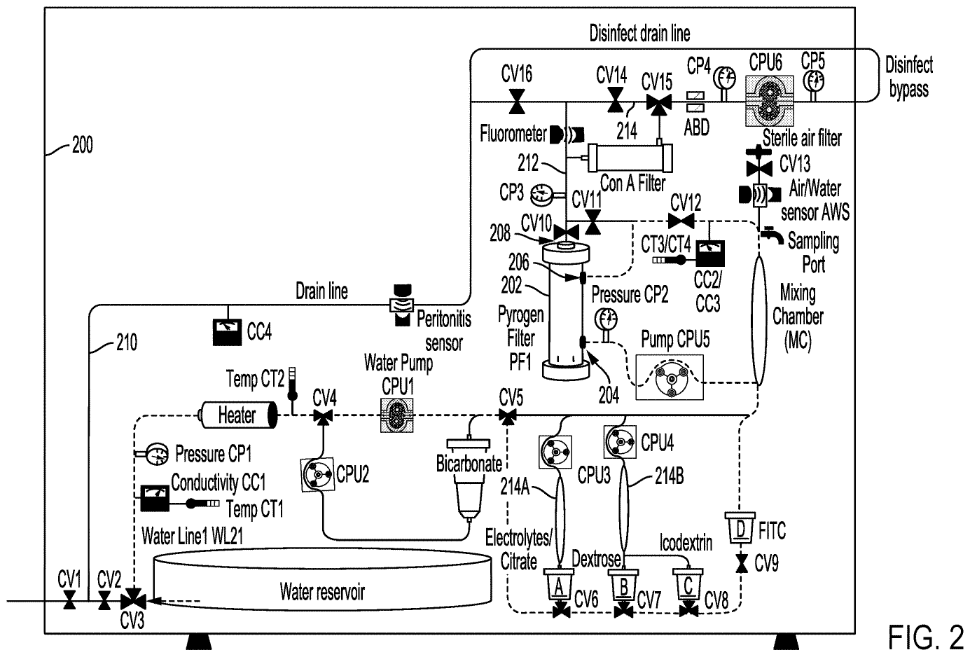

FIG. 2 is a schematic diagram of the system 200. In addition to the components shown in FIG. 1, the system 200 includes a mixing chamber MC, pumps CPU1-6, and a filter cartridge 202. The filter cartridge 202 includes a pyrogen filter, an inlet 204, a bypass outlet 206, and a filtering outlet 208. The system 200 also includes a fluorometer, valves CV1-16, and tubing lines that provide fluid connections for the valves CV1-16 and the other components of the system 200. The system 200 also includes a water reservoir, temperature sensors CT1-4, conductivity sensors CC1-4, pressure sensors CP1-5, a heater, a container of bicarbonate, a container of electrolyte and citrate and/or acetate, a container of a simple sugar such as glucose or dextrose, a container of an osmotic agent such as icodextrin, a container of a fluorescent probe material such as fluorescein isothiocyanate (FITC), and a vessel taking the form of a Con A filter. In some examples, the fluorescent probe material can take the form of derivatives of rhodamine (TRITC), coumarin, or cyanine. Other fluorescent probe materials are possible.

The mixing chamber MC is expandable and collapsable such that the volume of the mixing chamber MC can increase and decrease to accommodate different volumes of dialysate or other liquids. This expandable nature of the mixing chamber MC eliminates the need for venting air from the mixing chamber when filling the mixing chamber MC with liquid.

The Con A filter has one or more surfaces (e.g., stacked plates or fibers) with a lectin such as Concanavalin A that is bound to the surfaces. The lectin can take the form of Lens culinaris, Pisum sativum, and/or Vicia ervilia as well. The Con A filter also contains a fluorescent probe material such as FITC and a polysaccharide (e.g. Dextran) that are bound to each other and to the lectin. The lectin has a binding affinity for glucose that is greater than the binding affinity of the lectin to the polysaccharide.

The system 200 includes a closed fluid loop that includes the mixing chamber MC, the pump CPU5, the inlet 204, the bypass outlet 206, and the valve CV12. The temperature sensors CT3/CT4 are configured to detect a temperature of a fluid flowing through the closed fluid loop.

The system 200 includes a drain line 210 that is connected to the pyrogen filter via the valve CV10, the fluorometer line 212, and the valve CV16. The system 200 also includes a patient line 214. The fluorometer line 212 connects the filtering outlet 208 to the patient line 214 via the valve CV14.

The system 200 includes an expandable dissolution chamber 214A, an expandable dissolution chamber 214B, a water reservoir, a container B comprising a simple sugar such as Dextrose, a container C comprising an osmotic agent such as Icodextrin, a container A comprising electrolytes and a citrate and/or an acetate, and a container D comprising FITC.

The system 200 is configured to operate in in a fluorometer diagnostic mode, a filter diagnostic mode, a dialysate production mode, a dialysate dispensing mode, and a glucose quantification mode.

FIG. 2 shows the system 200 conducting a process in which water and dissolved FITC are moved into the mixing chamber MC and circulated by the pump CPU5. That is, the pump CPU5 and/or the pump CPU1 move the water from the water reservoir through the container D such that the water and dissolved FITC move through the pump CPU5, into the inlet 204, out of the bypass outlet 206, through the valve CV12, and into the mixing chamber MC to fill and expand the mixing chamber MC with water and dissolved FITC.

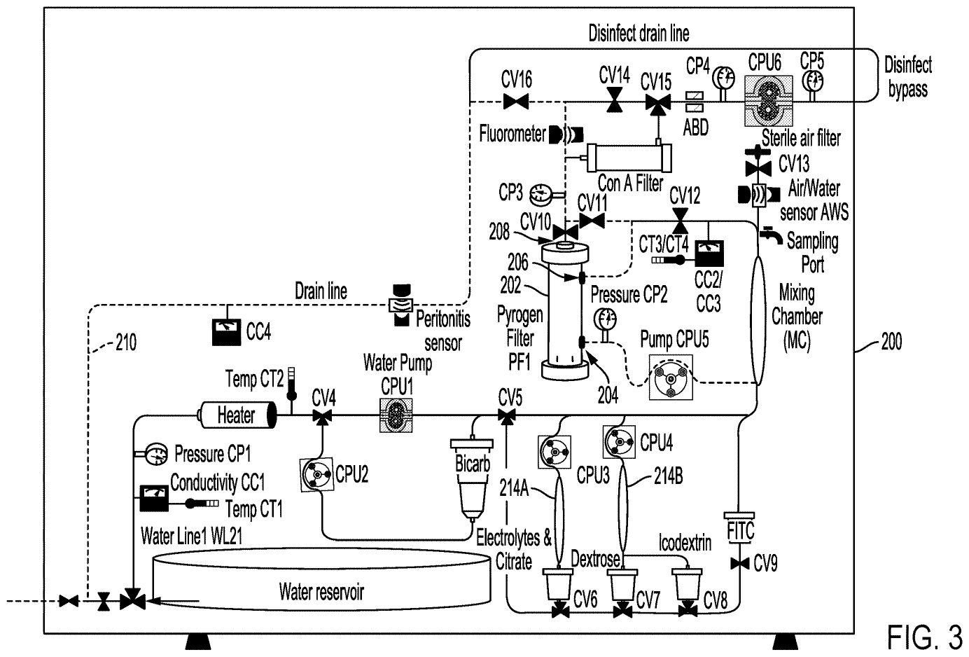

FIG. 3 shows the system 200 operating in the fluorometer diagnostic mode. The pump CPU5 moves a first portion of the water and a first portion of the fluorescent probe material (e.g., FTIC) from the mixing chamber MC, into the inlet 204, out of the bypass outlet 206, through the valve CV11, through the fluorometer, through the valve CV16, and to the drain line 210. The valve CV10 is closed and the valve CV11 is open so that the fluorescent probe material, which is formed of particles that are too large to pass through a working filter, are free to arrive at the fluorometer. The system 200 uses the fluorometer to detect the first portion of the fluorescent probe material dissolved in the first portion of the water as a diagnostic check to determine that the fluorometer is in working order. Next, the system 200 operates in the filter diagnostic mode in response to the system 200 passing the fluorometer diagnostic mode by detecting the FITC that was known to flow through the fluorometer.

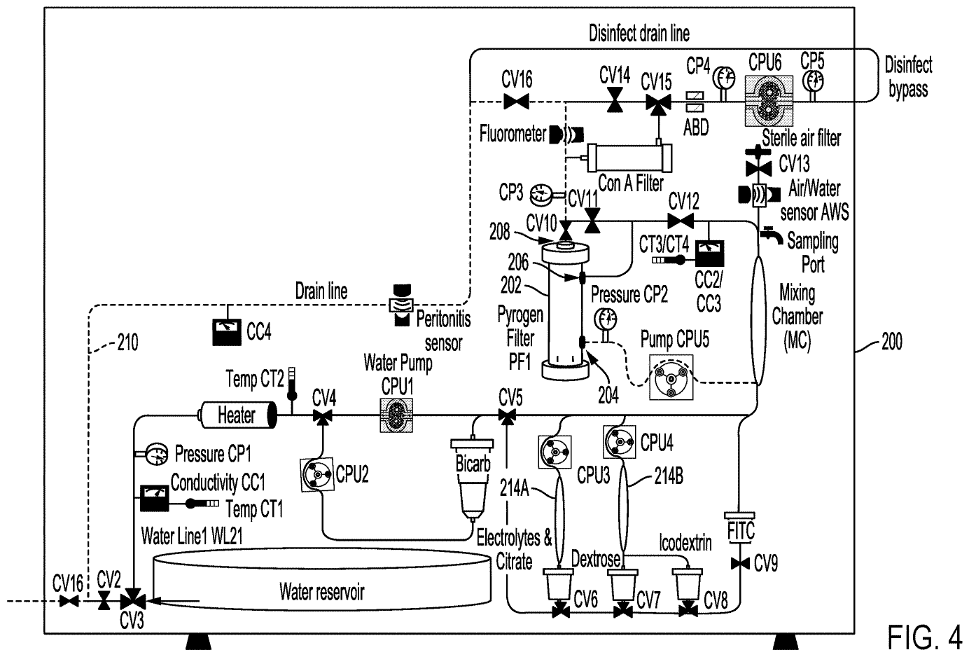

FIG. 4 shows the system 200 operating in the filter diagnostic mode in response to passing the fluorometer diagnostic mode. The pump CPU5 moves a second portion of the water and a second portion of the fluorescent probe material from the mixing chamber MC into the inlet 204 such that the second portion of the water moves through the filter, out of the filtering outlet 208, to the fluorometer, through the valve CV16, and to the drain line 210. As shown, the valve CV10 is open and the valve CV11 is closed to force most of the water entering the inlet 204 to pass through the filter instead of exiting through the bypass outlet 206. However, a small amount of water and the second portion of the fluorescent probe material that enters the inlet 204 is allowed to escape via the bypass outlet 206, through the slightly opened valve CV12, and back to the mixing chamber MC. When the filter is working properly, the fluorescent probe material particles are too large to pass through the filter. Next, the system 200 uses the fluorometer to determine that that less than a first threshold amount of the second portion of the fluorescent probe material passed through the filter with the second portion of the water. This means that the filter is working properly. As a result, the system 200 is ready to safely produce and dispense dialysate to a patient. The system 200 operates in the dialysate dispensing mode in response to determining that that less than a first threshold amount of the second portion of the fluorescent probe material passed through the filter with the second portion of the water.

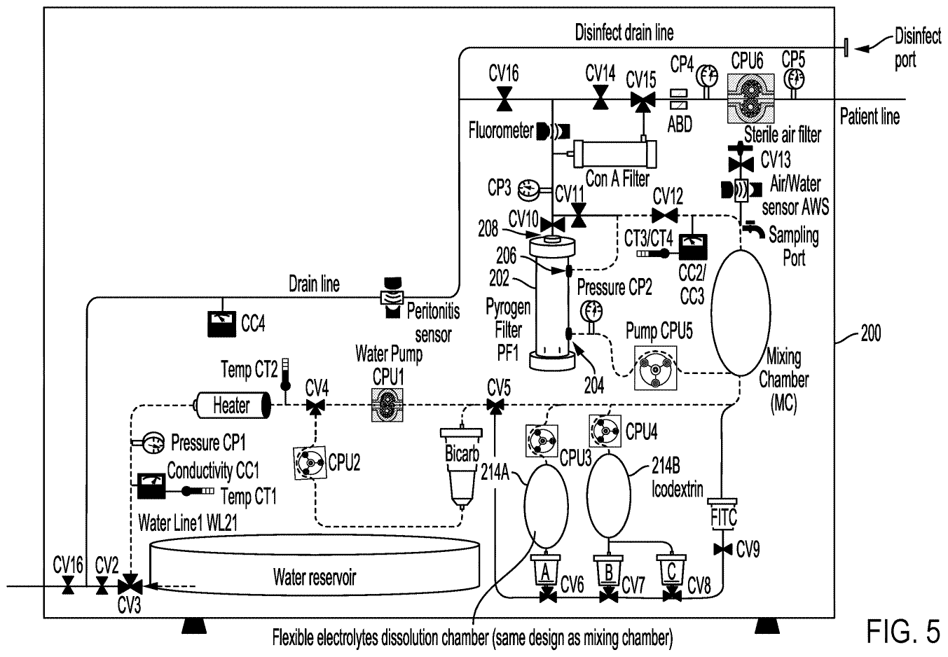

FIG. 5 shows the system 200 operating in the dialysate production mode. First, the water pump CPU1 moves water from the water reservoir through the container A, the container B, and the container C to fill the dissolution chamber 214A with a solution of water, electrolytes, and citrate and/or acetate and to fill the dissolution chamber 214B with a solution of water, a simple sugar, e.g. Dextrose, which is the standard dwell osmotic agent (which is distinct from the long dwell osmotic agent, icodextrin). Next, the water pump CPU1 moves water from the water reservoir through the valve CV5 to be mixed with the contents of the dissolution chamber 214A and the contents of the dissolution chamber 214B, forming dialysate in the mixing chamber MC.

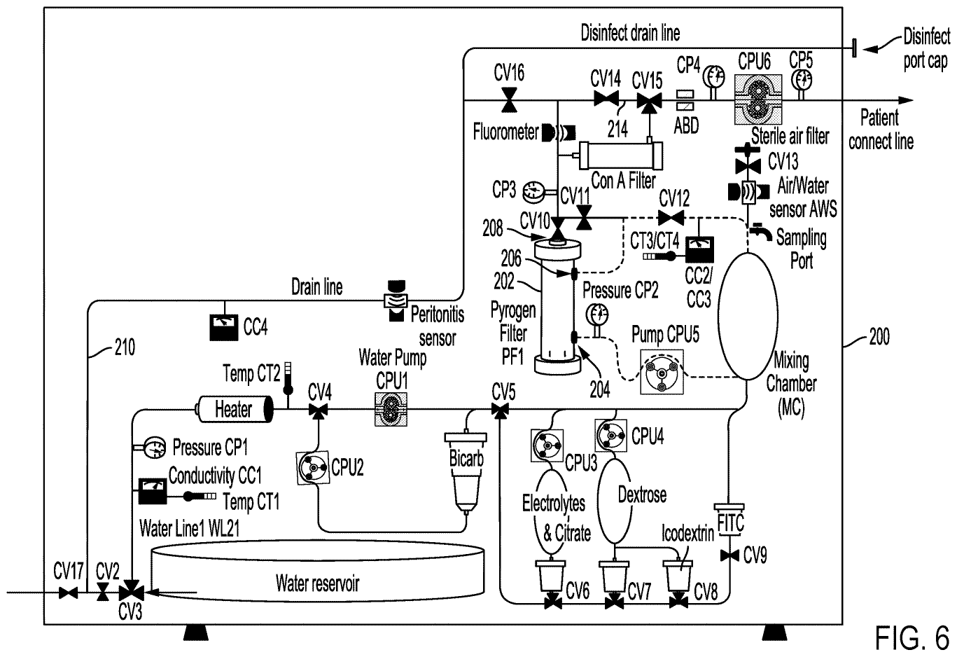

FIG. 6 shows the system 200 operating in the dialysate dispensing mode. After the mixing chamber MC is filled with dialysate as discussed above with reference to FIG. 5, the pump CPU5 moves the dialysate and a third portion of the fluorescent probe material from the mixing chamber MC into the inlet 204 such that the dialysate moves through the filter, out of the filtering outlet 208, and through the fluorometer.

If less than a threshold amount of the third portion of the fluorescent probe material is detected by the fluorometer, the dialysate is dispensed via the patient line 214. However, if more than the threshold amount of the third portion of the fluorescent probe material is detected by the fluorometer, the dialysate is dispensed via the drain line 210. The fluorescent probe material acts as a proxy for pyrogens due to their similar particle size. That is, as long as a negligible amount of the fluorescent probe material is detected by the fluorometer, it can be assumed that the filter is functioning properly and no significant amount of pyrogens are present in the filtered dialysate to be dispensed to the patient. However, if a non-negligible amount of the fluorescent probe material is detected by the fluorometer, it can be assumed that the filter is not functioning properly and a significant amount of pyrogens might be present in the filtered dialysate to be dispensed to the patient, which is why the dialysate is disposed of in this case.

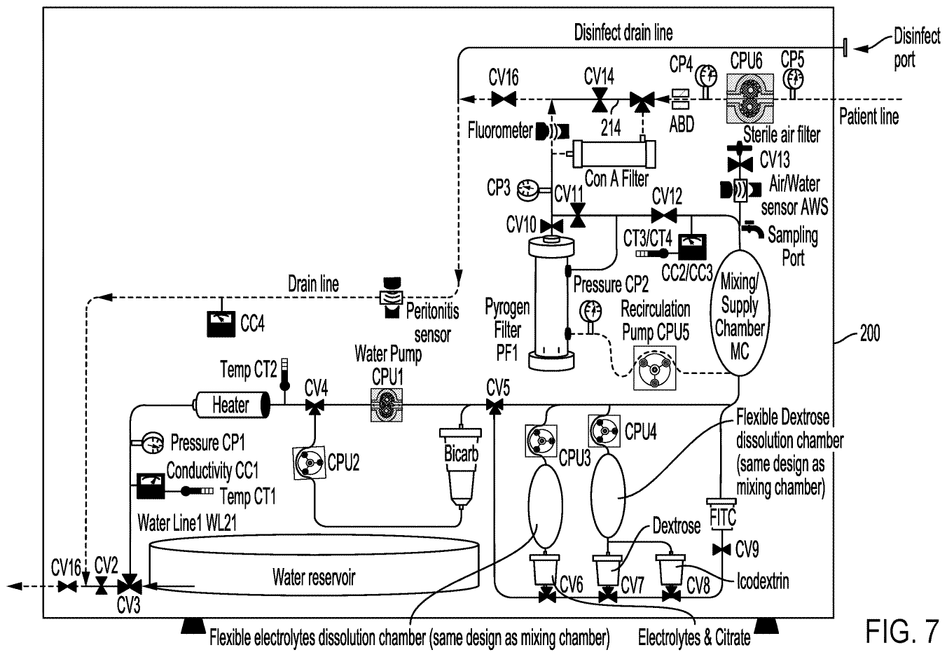

FIG. 7 shows the system 200 operating in the glucose quantification mode. The vessel taking the form of a Con A filter has (i) one or more surfaces (e.g., stacked plates and/or fibers) with a lectin such as Concanavalin A that is bound to the surfaces, and (ii) a fluorescent probe material and a polysaccharide that are bound to each other and to the lectin. The Con A filter receives the spent dialysate via the patient line 214, thereby binding the glucose present in the spent dialysate to the lectin and displacing the polysaccharide and the fluorescent probe material from the lectin. The pump CPU6 moves the spent dialysate, the polysaccharide, and the fluorescent probe material to the fluorometer. The fluorometer detects an amount of the fluorescent probe material within the spent dialysate. This detected amount of the fluorescent probe material is proportional to the amount of glucose remaining in the spent dialysate received from the patient.

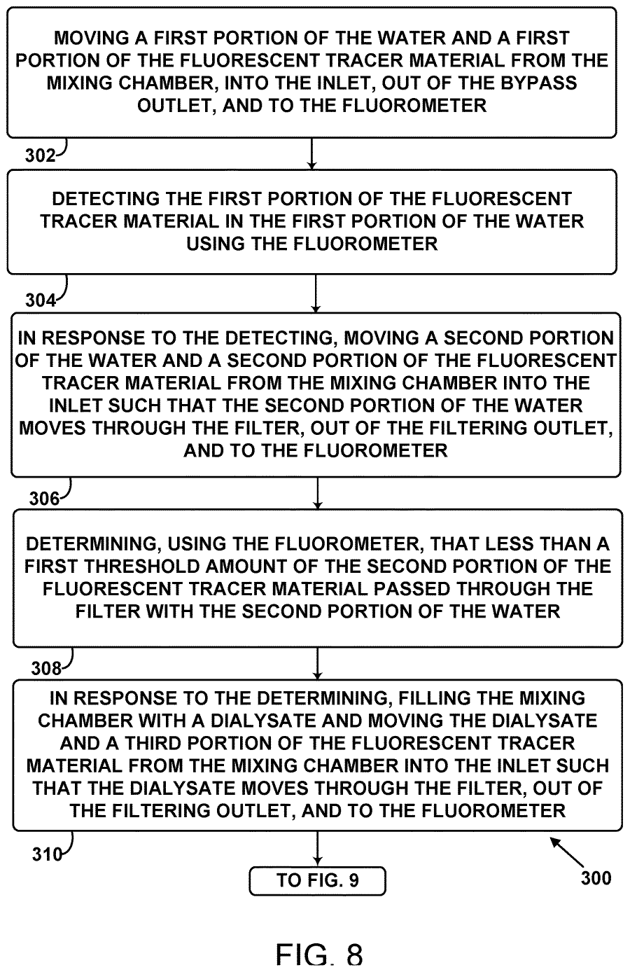

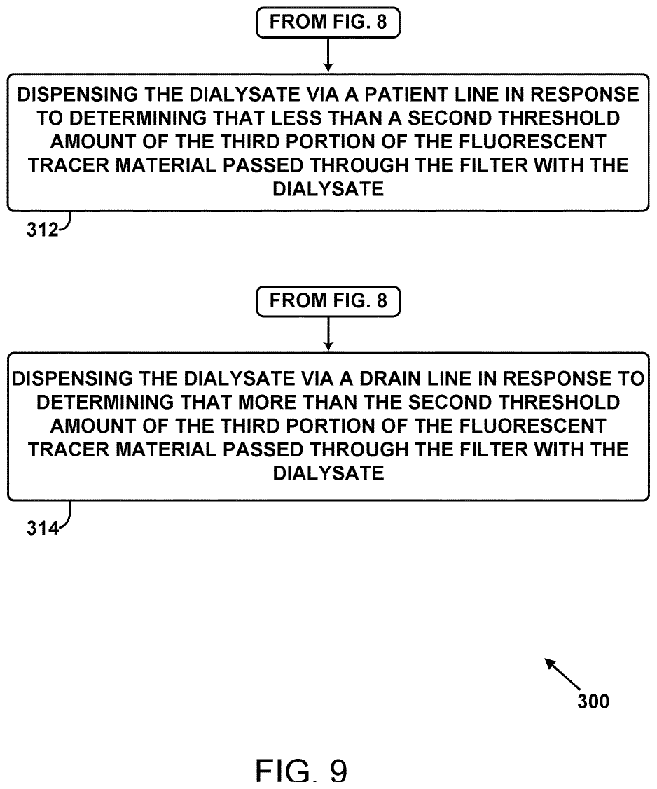

FIG. 8 and FIG. 9 are block diagrams of a method 300, which in some examples are performed by the system 200 and/or manually. As shown in FIGS. 8-9, the method 300 includes one or more operations, functions, or actions as illustrated by blocks 302, 304, 306, 308, 310, 312, and/or 314. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 302, the method 300 includes the system 200 moving a first portion of the water and a first portion of the fluorescent probe material from the mixing chamber MC, into the inlet 204, out of the bypass outlet 206, and to the fluorometer. Functionality related to block 302 is described above with reference to FIG. 3.

At block 304, the method 300 includes the system 200 detecting the first portion of the fluorescent probe material in the first portion of the water using the fluorometer. Functionality related to block 304 is described above with reference to FIG. 3.

At block 306, the method 300 includes, in response to the detecting, the system 200 moving a second portion of the water and a second portion of the fluorescent probe material from the mixing chamber MC into the inlet 204 such that the second portion of the water moves through the filter, out of the filtering outlet 208, and to the fluorometer. Functionality related to block 306 is described above with reference to FIG. 4.

At block 308, the method 300 includes the system 200 determining, using the fluorometer, that less than a first threshold amount of the second portion of the fluorescent probe material passed through the filter with the second portion of the water. Functionality related to block 308 is described above with reference to FIG. 4.

At block 310, the method 300 includes in response to the determining, the system 200 filling the mixing chamber MC with a dialysate and moving the dialysate and a third portion of the fluorescent probe material from the mixing chamber MC into the inlet 204 such that the dialysate moves through the filter, out of the filtering outlet 208, and to the fluorometer. Functionality related to block 310 is described above with reference to FIG. 5 and FIG. 6.

At block 312, the method 300 includes the system 200 dispensing the dialysate via a patient line 214 in response to determining that less than a second threshold amount of the third portion of the fluorescent probe material passed through the filter with the dialysate. Functionality related to block 312 is described above with reference to FIG. 6.

At block 314, the method 300 includes the system dispensing the dialysate via a drain line 210 in response to determining that more than the second threshold amount of the third portion of the fluorescent probe material passed through the filter with the dialysate. Although not explicitly shown in FIG. 6, functionality related to block 314 is described above with reference to FIG. 6.

### Further Example Embodiments

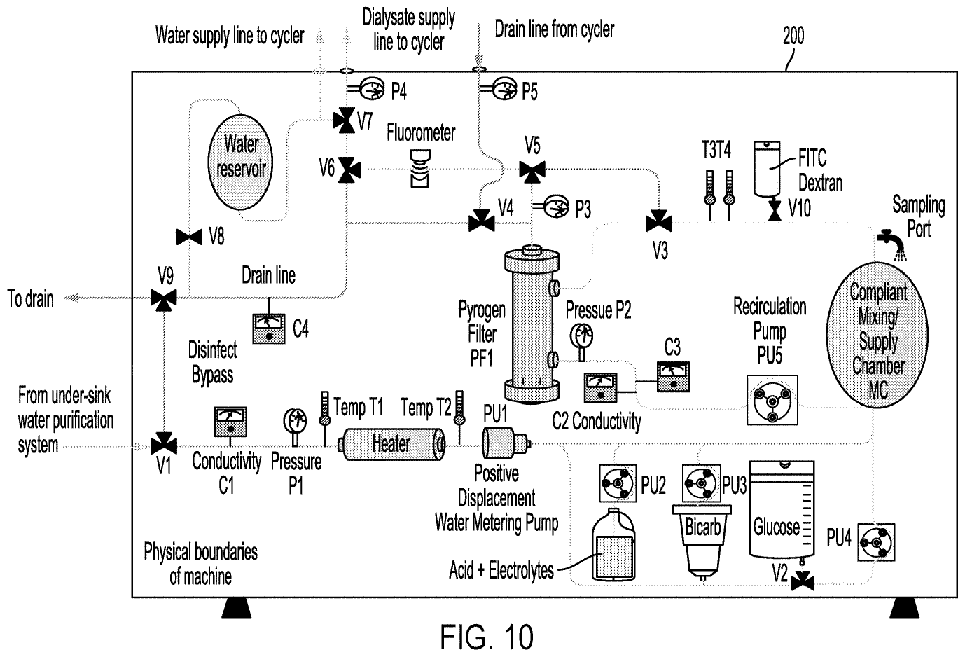

FIG. 10 depicts the system 200 which produces injectable quality peritoneal dialysate by metering purified water together with concentrates of Magnesium, Sodium, Calcium, bicarbonate, and Dextrose into a mixing chamber and ultra-purifying the homogenized solution through a depyrogenation filter. In this embodiment, a single depyrogenation filter is used rather than two depyrogenation filters. A recirculation circuit is constructed by forming a closed loop of tubing that exits the mixing chamber at its bottom, is connected to a recirculation pump (PU5), then flows through the pyrogen filter (PF1), three way valve V3, and then back into the mixing chamber. Included in this recirculation loop are redundant conductivity and temperature sensors which are used to help assure that the composition of the mixed dialysate is correct and that it is at the prescribed temperature before allowing it to proceed to a patient.

Valve V3 connects this recirculation loop to the drain line via valves V5, V6 and V9. Located in the tubing segment between valve V5 and V6 is an in-line, flow-through fluorometer which is designed to detect fluorescent marker molecules, one example of which is fluorescein isothiocyanate (FITC). One advantage of FITC is that it is available as a conjugate to various molecular weight sizes of dextran. Some of these conjugates are of molecular sizes (e.g. >70,000 Daltons) that are too large to pass through the intact membrane of pyrogen filters.

Included in the recirculation loop is valve V10 which isolates a container of 70K Dalton FITC dextran from the loop. The initial step in preparing for a dialysis treatment is to flush all fluid pathways with ultrapure water. At the end of that phase, all fluid paths are filled with water and pump PU5 continues to recirculate that water around the loop. At that point, a sufficient amount of the FITC dextran is allowed to enter the recirculation loop by opening valve V10 such that the resultant concentration can be reliably detected by the in-line fluorometer.

A T₀ test is next performed to verify that the fluorometer is operating correctly. This is accomplished by allowing some of the water in the recirculation loop to exit via valve V3 and pass through valve V5 and past the fluorometer. If the fluorometer is operating as expected, it will register a fluorescence signal from the FITC dextran and will pass the T₀ test.

Next, a T₀ test will be performed on the pyrogen filter to verify that its membrane is intact. This is accomplished by closing valve V3 and opening valve V5 to the product water output port of the pyrogen filter such that the water in the recirculation loop will be pushed through the membrane of the pyrogen filter (the volume of the compliant mixing chamber decreases as fluid is removed from the recirculation loop). This product water flows past valve V5 and then past the fluorometer before flowing past valve V6 and V9 and then down the drain line.

In this case, the expectation is that no fluorescence will be detected by the fluorometer since the FITC dextran contained in the recirculation loop is too large to pass through an intact pyrogen filter membrane. If any FITC dextran were able to penetrate the membrane and register a positive reading for fluorescence by the fluorometer, it could only mean that there was a broken fiber or other perforation in the membrane and it would fail the test requiring replacement before another dialysis treatment would be permitted.

Once in the recirculation loop, the FITC dextran will remain throughout the treatment. Before the dialysate concentrates are metered into the mixing chamber to create the prescribed final formulation of dialysate, the majority, but not all, of the water previously contained in the recirculation loop will be sent to drain through the pyrogen filter membrane all the while being surveilled by the fluorometer. A small amount of water is retained in the loop so that the FITC dextran can be retained there while still in solution. The amount of water retained is known with the required amount of accuracy since the volume of the recirculation loop is known when the mixing chamber is at its minimum volume (compliance is fully minimized). This volume of water is taken into consideration (in software) in directing how much of the concentrates and additional water to bring into the mixing chamber to result in the correct final dialysate composition.

In this way, FITC dextran is generally always present in the recirculation loop and, consequently, the integrity of the pyrogen filter membrane is continuously supported before, during, and after a dialysis treatment is performed, thereby continuously supporting the safety of the patient. By so doing, the requirement for a second, redundant pyrogen filter can be eliminated as well as the need to perform any other method of verifying membrane integrity such as an air pressure decay test.

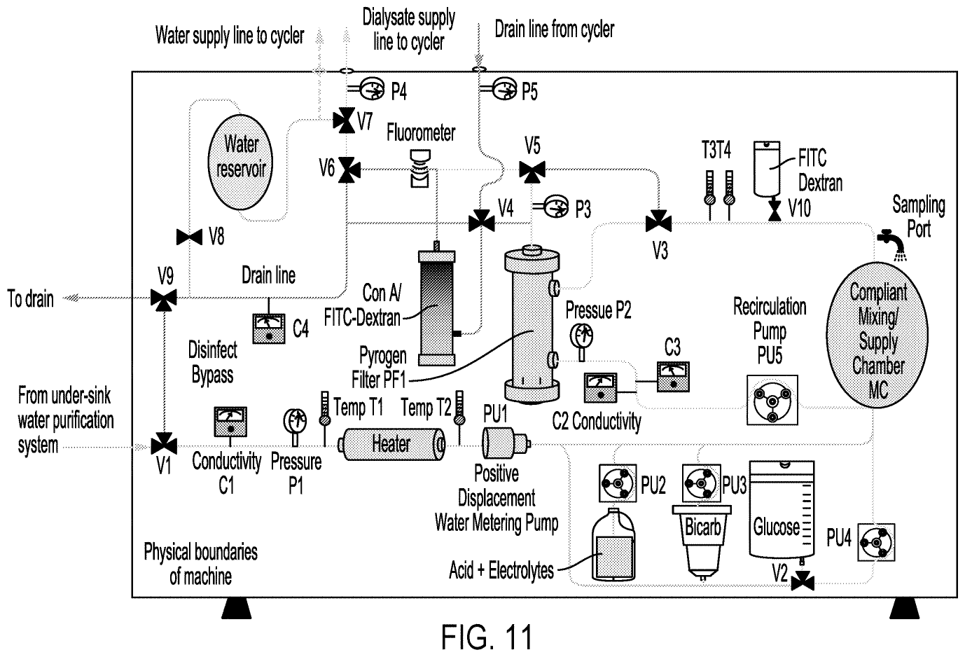

FITC dextran along with the in-line fluorometer can also be utilized to create a method of quantifying the amount of glucose resident in the effluent dialysate being drained from the patient during each drain phase. However, in this embodiment, the source of FITC dextran is not the same container that is connected to the recirculation loop. Rather, it is conjugated to the surface of a container located in an extension of the drain line originating from the cycler as shown in FIG. 11 between valve V4 and the fluorometer.

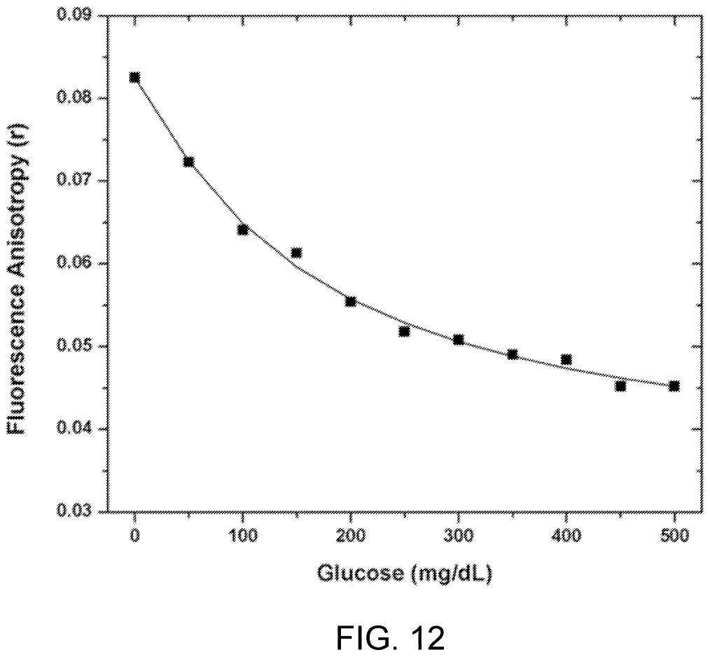

FITC dextran can be bound to a lectin such as Concanavalin A which, in turn, can be bound to some surface over which the effluent dialysate can be perfused. This surface could be the hollow fibers of a filtration device similar in construction to pyrogen filters or hemodialyzers. This technique is based upon a competitive binding assay. FITC dextran has a certain binding strength to Con A that is lower than the binding strength of monomeric glucose. Consequently, as effluent dialysate containing glucose enters the hollow fibers (for example) where the FITC dextran is bound, the glucose will competitively displace the FITC dextran sending it downstream and past the in-line fluorometer. The strength of the fluorescence signal has been shown to be accurately proportional to the concentration of glucose in the perfused solution as illustrated FIG. 12.

Using this type of conversion of fluorescence to glucose concentration, the quantification of the total amount of glucose drained from the patient is possible. When the drained amount of glucose is then subtracted from the instilled amount residing in the fresh dialysate from the previous fill cycle, the amount of glucose absorbed by the patient is the result.

### Example Enumerated Embodiments (EEEs)

EEE 1 is a system for producing dialysate, the system comprising: a mixing chamber; a pump; a filter cartridge comprising a filter, an inlet, a bypass outlet, and a filtering outlet; a fluorometer; one or more valves; and one or more tubing lines, wherein the system is configured to operate in a fluorometer diagnostic mode, a filter diagnostic mode, and a dialysate dispensing mode, wherein in the fluorometer diagnostic mode the one or more valves and the one or more tubing lines are configured to allow the pump to move water and a fluorescent probe material from the mixing chamber, into the inlet, out of the bypass outlet, and to the fluorometer, wherein in the filter diagnostic mode the one or more valves and the one or more tubing lines are configured to allow the pump to move the water and the fluorescent probe material from the mixing chamber into the inlet such that (i) a first portion of the water and the fluorescent probe material moves out of the bypass outlet and back to the mixing chamber and (ii) a second portion of the water moves through the filter, out of the filtering outlet, and to the fluorometer, and wherein in the dialysate dispensing mode the one or more valves and the one or more tubing lines are configured to allow the pump to move a dialysate and the fluorescent probe material from the mixing chamber into the inlet such that (i) a first portion of the dialysate and the fluorescent probe material moves out of the bypass outlet and back to the mixing chamber and (ii) a second portion of the dialysate moves through the filter, out of the filtering outlet, and to the fluorometer.

EEE 2 is the system of EEE 1, wherein the mixing chamber is expandable.

EEE 3 is the system of any one of EEEs 1-2, further comprising a vessel having (i) a surface with a lectin that is bound thereto, and (ii) a fluorescent probe material and a polysaccharide that are bound to each other and to the lectin, wherein the system is further configured to operate in a glucose quantification mode wherein the one or more valves and the one or more tubing lines are configured to allow (i) the vessel to receive spent dialysate containing glucose, thereby binding the glucose to the lectin and displacing the polysaccharide and the fluorescent probe material from the lectin, and (ii) the spent dialysate, the polysaccharide, and the fluorescent probe material to move to the fluorometer.

EEE 4 is the system of EEE 3, wherein the lectin comprises Concanavalin A.

EEE 5 is the system of EEE 3, wherein the lectin has a binding affinity for glucose greater than the binding affinity of the lectin to the polysaccharide.

EEE 6 is the system of any one of EEEs 1-5, wherein the filter comprises a pyrogen filter.

EEE 7 is the system of any one of EEEs 1-6, wherein the fluorescent probe material comprises fluorescein isothiocyanate.

EEE 8 is the system of any one of EEEs 1-7, wherein the system includes a closed fluid loop that includes the mixing chamber, the pump, the inlet, and the bypass outlet.

EEE 9 is the system of EEE 8, further comprising one or more temperature sensors configured to detect a temperature of a fluid flowing through the closed fluid loop.

EEE 10 is the system of any one of EEEs 1-9, wherein the one or more tubing lines comprise a drain line, wherein the one or more valves and the one or more tubing lines connect the filter to the drain line.

EEE 11 is the system of EEE 10, wherein the one or more tubing lines comprise a fluorometer line that connects the filter to the drain line via the one or more valves, wherein the fluorometer line passes through the fluorometer.

EEE 12 is the system of EEE 11, wherein the one or more tubing lines comprise a patient line, wherein the fluorometer line connects the filter to the patient line via the one or more valves.

EEE 13 is the system of any one of EEEs 1-12, further comprising a first expandable dissolution chamber, a water reservoir, a first container comprising a simple sugar, and a second container comprising an osmotic agent, wherein the system is configured to operate in a dialysate production mode wherein the one or more valves and the one or more tubing lines are configured to allow water to flow from the water reservoir through the first container and the second container to move the water, the simple sugar, and the osmotic agent into the first expandable dissolution chamber.

EEE 14 is the system of EEE 13, further comprising a second expandable dissolution chamber and a third container comprising electrolytes and a citrate and/or an acetate, wherein in the dialysate production mode the system is further configured to allow the water to flow from the water reservoir through the third container to move the water, the electrolytes, and the citrate and/or the acetate into the second expandable dissolution chamber.

EEE 15 is the system of EEE 14, wherein in the dialysate production mode the system is further configured to allow (i) the water, the electrolytes, and the citrate and/or the acetate to move from the second expandable dissolution chamber to the mixing chamber, and (ii) the water, the osmotic agent, and the simple sugar to move from the first expandable dissolution chamber to the mixing chamber.

EEE 16 is a method of operating the system of any one of EEEs 1-15 to dispense dialysate, the method comprising: moving a first portion of the water and a first portion of the fluorescent probe material from the mixing chamber, into the inlet, out of the bypass outlet, and to the fluorometer; detecting the first portion of the fluorescent probe material in the first portion of the water using the fluorometer; in response to the detecting, moving a second portion of the water and a second portion of the fluorescent probe material from the mixing chamber into the inlet such that the second portion of the water moves through the filter, out of the filtering outlet, and to the fluorometer; determining, using the fluorometer, that less than a first threshold amount of the second portion of the fluorescent probe material passed through the filter with the second portion of the water; in response to the determining, filling the mixing chamber with a dialysate and moving the dialysate and a third portion of the fluorescent probe material from the mixing chamber into the inlet such that the dialysate moves through the filter, out of the filtering outlet, and to the fluorometer; and dispensing the dialysate via a patient line in response to determining that less than a second threshold amount of the third portion of the fluorescent probe material passed through the filter with the dialysate; or dispensing the dialysate via a drain line in response to determining that more than the second threshold amount of the third portion of the fluorescent probe material passed through the filter with the dialysate.

EEE 17 is the method of EEE 16, further comprising filling the mixing chamber with the water and the fluorescent probe material prior to moving the first portion of the water and the first portion of the fluorescent probe material.

EEE 18 is the method of any one of EEEs 16-17, further comprising: the vessel receiving the spent dialysate, thereby binding the glucose to the lectin and displacing the polysaccharide and the fluorescent probe material from the lectin; moving the spent dialysate, the polysaccharide, and the fluorescent probe material to the fluorometer; and detecting an amount of the fluorescent probe material within the spent dialysate using the fluorometer.

EEE 19 is the method of any one of EEEs 16-18, wherein the filter comprises a pyrogen filter.

EEE 20 is the method of any one of EEEs 16-19, wherein the fluorescent probe material comprises fluorescein isothiocyanate.

EEE 21 is the method of any one of EEEs 16-20, further comprising flowing water from the water reservoir through the first container and the second container to move the water, the simple sugar, and the osmotic agent into the first expandable dissolution chamber.

EEE 22 is the method of EEE 22, further comprising flowing water from the water reservoir through the third container to move the water, the electrolytes, and the citrate and/or the acetate into the second expandable dissolution chamber.

EEE 23 is the method of EEE 22, further comprising moving (i) the water, the electrolytes, and the citrate and/or the acetate from the second expandable dissolution chamber to the mixing chamber, and (ii) the water, the osmotic agent, and the simple sugar from the first expandable dissolution chamber to the mixing chamber.

EEE 24 is A non-transitory computer readable medium storing instructions that, when executed by a computing device, cause the computing device to perform the method of any one of EEEs 16-23.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1. A system for producing dialysate, the system comprising:
a mixing chamber;
a pump;
a filter cartridge comprising a filter, an inlet, a bypass outlet, and a filtering outlet;
a fluorometer;
one or more valves; and
one or more tubing lines, wherein the system is configured to operate in a fluorometer diagnostic mode, a filter diagnostic mode, and a dialysate dispensing mode,
wherein in the fluorometer diagnostic mode the one or more valves and the one or more tubing lines are configured to allow the pump to move water and a fluorescent probe material from the mixing chamber, into the inlet, out of the bypass outlet, and to the fluorometer,
wherein in the filter diagnostic mode the one or more valves and the one or more tubing lines are configured to allow the pump to move the water and the fluorescent probe material from the mixing chamber into the inlet such that (i) a first portion of the water and the fluorescent probe material moves out of the bypass outlet and back to the mixing chamber and (ii) a second portion of the water moves through the filter, out of the filtering outlet, and to the fluorometer, and
wherein in the dialysate dispensing mode the one or more valves and the one or more tubing lines are configured to allow the pump to move a dialysate and the fluorescent probe material from the mixing chamber into the inlet such that (i) a first portion of the dialysate and the fluorescent probe material moves out of the bypass outlet and back to the mixing chamber and (ii) a second portion of the dialysate moves through the filter, out of the filtering outlet, and to the fluorometer.

2. The system of claim 1, wherein the mixing chamber is expandable.

3. The system of claim 1, further comprising a vessel having (i) a surface with a lectin that is bound thereto, and (ii) a fluorescent probe material and a polysaccharide that are bound to each other and to the lectin, wherein the system is further configured to operate in a glucose quantification mode wherein the one or more valves and the one or more tubing lines are configured to allow (i) the vessel to receive spent dialysate containing glucose, thereby binding the glucose to the lectin and displacing the polysaccharide and the fluorescent probe material from the lectin, and (ii) the spent dialysate, the polysaccharide, and the fluorescent probe material to move to the fluorometer.

4. The system of claim 3, wherein the lectin comprises Concanavalin A.

5. The system of claim 3, wherein the lectin has a binding affinity for glucose greater than the binding affinity of the lectin to the polysaccharide.

6. The system of claim 1, wherein the filter comprises a pyrogen filter and/or the fluorescent probe material comprises fluorescein isothiocyanate.

7. (canceled)

8. The system of claim 1, wherein the system includes a closed fluid loop that includes the mixing chamber, the pump, the inlet, and the bypass outlet.

9. The system of claim 8, further comprising one or more temperature sensors configured to detect a temperature of a fluid flowing through the closed fluid loop.

10. The system of claim 1, wherein the one or more tubing lines comprise a drain line, wherein the one or more valves and the one or more tubing lines connect the filter to the drain line.

11. The system of claim 10, wherein the one or more tubing lines comprise a fluorometer line that connects the filter to the drain line via the one or more valves, wherein the fluorometer line passes through the fluorometer.

12. The system of claim 11, wherein the one or more tubing lines comprise a patient line, wherein the fluorometer line connects the filter to the patient line via the one or more valves.

13. The system of claim 1, further comprising a first expandable dissolution chamber, a water reservoir, a first container comprising a simple sugar, and a second container comprising an osmotic agent, wherein the system is configured to operate in a dialysate production mode wherein the one or more valves and the one or more tubing lines are configured to allow water to flow from the water reservoir through the first container and the second container to move the water, the simple sugar, and the osmotic agent into the first expandable dissolution chamber.

14. The system of claim 13, further comprising a second expandable dissolution chamber and a third container comprising electrolytes and a citrate and/or an acetate, wherein in the dialysate production mode the system is further configured to allow the water to flow from the water reservoir through the third container to move the water, the electrolytes, and the citrate and/or the acetate into the second expandable dissolution chamber.

15. The system of claim 14, wherein in the dialysate production mode the system is further configured to allow (i) the water, the electrolytes, and the citrate and/or the acetate to move from the second expandable dissolution chamber to the mixing chamber, and (ii) the water, the osmotic agent, and the simple sugar to move from the first expandable dissolution chamber to the mixing chamber.

16. A method of operating the system of claim 1 to dispense dialysate, the method comprising:
moving a first portion of the water and a first portion of the fluorescent probe material from the mixing chamber, into the inlet, out of the bypass outlet, and to the fluorometer;
detecting the first portion of the fluorescent probe material in the first portion of the water using the fluorometer;
in response to the detecting, moving a second portion of the water and a second portion of the fluorescent probe material from the mixing chamber into the inlet such that the second portion of the water moves through the filter, out of the filtering outlet, and to the fluorometer;
determining, using the fluorometer, that less than a first threshold amount of the second portion of the fluorescent probe material passed through the filter with the second portion of the water;
in response to the determining, filling the mixing chamber with a dialysate and moving the dialysate and a third portion of the fluorescent probe material from the mixing chamber into the inlet such that the dialysate moves through the filter, out of the filtering outlet, and to the fluorometer; and
dispensing the dialysate via a patient line in response to determining that less than a second threshold amount of the third portion of the fluorescent probe material passed through the filter with the dialysate; or
dispensing the dialysate via a drain line in response to determining that more than the second threshold amount of the third portion of the fluorescent probe material passed through the filter with the dialysate.

17. The method of claim 16, further comprising filling the mixing chamber with the water and the fluorescent probe material prior to moving the first portion of the water and the first portion of the fluorescent probe material.

18. The method of claim 16, further comprising:
the vessel receiving the spent dialysate, thereby binding the glucose to the lectin and displacing the polysaccharide and the fluorescent probe material from the lectin;
moving the spent dialysate, the polysaccharide, and the fluorescent probe material to the fluorometer; and
detecting an amount of the fluorescent probe material within the spent dialysate using the fluorometer.

19. The method of claim 16, wherein the filter comprises a pyrogen filter and/or the fluorescent probe material comprises fluorescein isothiocyanate.

20. (canceled)

21. The method of claim 16, further comprising flowing water from the water reservoir through the first container and the second container to move the water, the simple sugar, and the osmotic agent into the first expandable dissolution chamber.

22-23. (canceled)

24. A non-transitory computer readable medium storing instructions that, when executed by a computing device, cause the computing device to perform the method of claim 16.
